(19) United States
(12) Patent Application Publication
(10) Pub. No.: US 2021/0192911 A1
(43) Pub. Date: Jun. 24, 2021

(54) FOREST MONITORING SYSTEM AND METHOD

(71) Applicants: Esthevan Augusto GOES GASPAROTO, Sao Paulo (BR); Emily Tsiemi SHINZATO, Sao Paulo (BR)

(72) Inventors: Esthevan Augusto GOES GASPAROTO, Sao Paulo (BR); Emily Tsiemi SHINZATO, Sao Paulo (BR)

(21) Appl. No.: 16/075,940

(22) PCT Filed: Feb. 6, 2017
(86) PCT No.: PCT/BR2017/000013

§ 371 Date: Aug. 6, 2018

(30) Foreign Application Priority Data
Feb. 5, 2016 (BR) BR1020160027055
Feb. 6, 2017 (BR) BR1020170023974

(51) Int. Cl. G08B 17/00; G08B 17/113

(52) U.S. Cl. CPC G08B 17/005; G08B 17/113

(57) ABSTRACT

Described is a forest monitoring system and method. Monitoring is performed continuously and remotely and evaluates factors such as growth and health, as well as climate and environment variables in forests, wooded surfaces or thus identified surfaces, using wireless data transfer.

### FIELD OF THE INVENTION

This invention pertains to the field of human and engineering needs, more specifically, to the field of botanic and measuring instruments, and refers to a system and method for forest monitoring.

Said monitoring takes place remotely and continuously and evaluates factors such as growth and health, as well as climatic and environmental variables in forests, wooded areas or thus characterized surfaces using wireless data transfer.

### BACKGROUND OF THE INVENTION

Currently, forest monitoring is carried out by means of a data collection carried out by a team of people who displaces to the area of interest and who manually collects data of the diameter of the trees, using measuring tape or bevel—pachymeter, tools with precision of millimeters and subject to reading and manipulating errors during the operation.

Devices such as electronic or mechanical dendrometers were developed with the purpose of meeting the needs of scientific research in the forest area. However, none of these apparatuses has the ability to measure large perimeter variations in trees over time or to function uninterruptedly for five or even more typical demands of the forest industry.

It further worth to highlight that during this activity, there is no collection of data of environmental variables, only as to diameter data of the trees.

Accordingly, this invention provides a system and a method that allow monitoring the growth and quality of the forest in real time and anywhere in the world. Therefore, the need for manual measurement of the forest is eliminated, reducing the possibility of work accidents and reducing displacement and transportation costs.

### STATE OF THE ART

Some prior art documents describe technologies related to systems and devices for measuring plants and trees.

Documents U.S. Pat. Nos. 4,549,355 and 7,398,602B2, respectively entitled “ELECTRONIC DENDROMETER” and “PRECISION DENDROMETER,” address to precision electronic dendrometers for measuring plants. In US20140360037A1, titled “DENDROMETER,” the precision electronic dendrometer as described presents wireless communication.

Documents US54G6715 and U.S. Pat. No. 8,640,460B1, respectively entitled “TAPE MEASURE DEVICE” and “MEASURING DEVICE AND METHOD,” describe a measuring tape for measuring circumferences.

Document U.S. Pat. No. 6,009,631, entitled “GAUGE FOR MEASURING CHANGES IN THE LENGTH OF A PERIMETER,” is an apparatus for measuring changes in the length of a given perimeter, while document U.S. Pat. No. 7,146,743B2, entitled “LENGTH MEASURING INSTRUMENT,” refers to an electronic instrument for measuring circumferences.

Document U.S. Pat. No. 8,174,397B2, entitled “MULTI-FUNCTIONAL MONITOR,” describes a multi-functional monitor for measuring environmental variables.

Document US201501160092A1, entitled “GIRTH MEASURING. DEVICE AND METHOD FOR. MEASURING GIRTH OF TREE, AND WIRELESS COMMUNICATION TAG APPARATUS INCLUDING GIRTH MEASURING DEVICE,” is a wireless measuring device for circumference measuring with wireless communication.

It is important to highlight that the technologies listed above have limited monitoring capacity in the long term or over five years and do not monitor environmental variables.

In addition, they have limited capacity to operate by wireless communication, have limited operating range (Δ≤300 mm) and do not monitor the actual measurement (only growth variation in relation to a starting point).

Therefore, even if solutions are proposed, such solutions admit the possibility of reading errors, as well as interpretation and manipulation of the obtained data, this being a severe deficiency in the forest monitoring and measurement.

In view of the above, the invention described herein allows the association of diametral growth measures of the trees or vegetation cover constituting the forest, as well as climatic and environmental variables, thus allowing the modeling of forest cover growth in the past, present, and future in a reliable and consistent way.

### BRIEF DESCRIPTION OF THE INVENTION

The present invention describes a system and method for forest monitoring, wherein said system consists of two devices—dendrometers (1) and a concentrator (19)—which, when connected to each other, form a smart parcel (34) that performs the measurement, processing, storage and transmission of informations, such as trunk diameter, morphological, biological and health status of the trees, air humidity and temperature, among others.

### BRIEF DESCRIPTION OF THE FIGURES

In order to obtain full and complete visualization of the object of this invention, there are presented figures to which reference is made, as follows.

FIG. 1 is the illustration of the dendrometer (1).

FIG. 2 is the representation of the logical scheme of the dendrometer (1).

FIG. 3 is the illustration of the concentrator (19).

FIG. 4 is the representation of the logic diagram of the concentrator (19).

FIG. 5 shows the network formed by the numerous dendrometers (1) connected to a concentrator (19).

FIG. 6 shows a network of concentrators (19).

### DETAILED DESCRIPTION OF THE INVENTION

The present invention describes a system and method for forest monitoring, wherein said system consists of two devices—dendrometers (1) and a concentrator (19)—which, when connected to each other, form a smart plot (34) that performs the measurement, processing, storage and transmission of information, such as trunk diameter, morphological, biological status and health. of trees, air humidity and temperature, fire risk, level of weed competition, among others.

The dendrometers (1) comprise a set of sensors capable of monitoring and measuring the diametral growth of trees, as well as their physical (morphological, biological and health) status. More specifically, the dendrometer consists of:
A cable (2);
A stressing band spring (3);
A smaller gear (4);
A gear (5);
A rotary sensor (6);
An upper part of the casing (7);
A lower part of the casing (8);
A cable fixing pin (9);
A printed circuit board (10);
A battery (11);
A spring fixing central pin (13);
A microcontroller or microprocessor (14);
An antenna (15);
An EEPROM memory (16);
A digital analog converter (7); and
A temperature sensor (18).

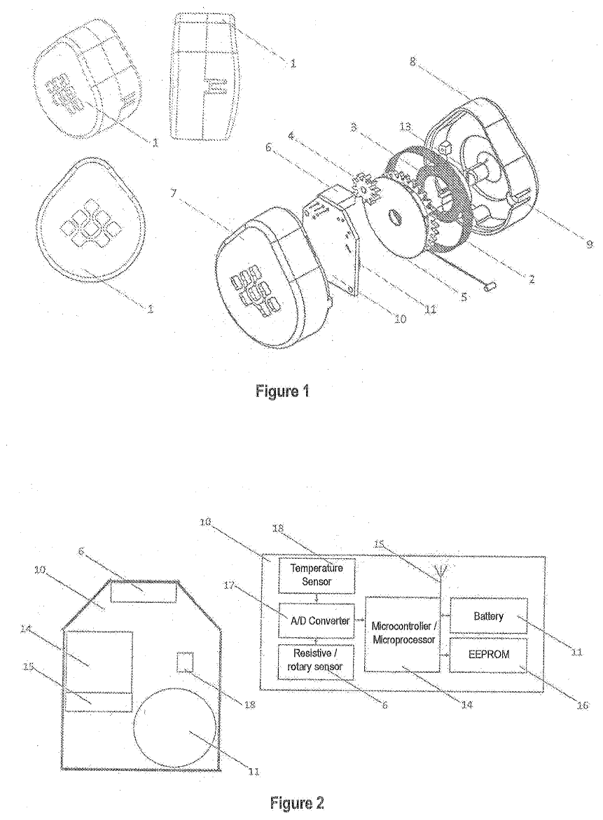

As can be noted in FIGS. 1 and 2, the dendrometer (1) contains a cable (2) made of metal or polymeric material, surrounding the trunk of the tree (12), thereby fixing the device thereto. As the trunk diameter of the tree increases, this cable (2) unwinds from the gear (5) which is constantly stressed by the spring (3).

The cable (2) is spool wounded in the gear (5) and, when unwound, moves the gear (5) that, in turn, moves the smaller gear (4).

When moved (i.e., with the stressing of the tape), said smaller gear (4) transfers this movement to the rotary sensor (6), which may be resistive, capacitive, inductive or of pulse counting.

The stressing band (3) can be a band spring, which exerts sufficient stress on the cable so that the entire dendrometer (1) remains secured to the trunk by means of static friction, without impairing however the physiological development of the tree.

The printed circuit board (10) is fed by a battery (11) containing the rotary sensor (6) and a temperature sensor (18) to a digital analog converter (17) capable of converting the analog sensor reading into digital values.

The temperature sensor (18) is used to correct and compensate for system expansion with respect to the temperature.

The digitized signals are processed by the microcontroller or microprocessor (14) that, in turn, stores the informations in the EEPROM memory (6) and transmits them via the antenna (15) using radio frequency methodology, preferably Bluetooth Low Energy (BLE), Long Range Radio (LoRa) or other methodology of Low-Power Wide Area Network (LPWAN) for the concentrator (19).

The upper part of the casing (7) and the lower part of the casing (8) are of polymeric material with protection against ultraviolet rays, also protecting the equipment from insect entry and moisture.

The concentrator (19), which is attached to the tree by means of glue, nails, springs or braces, is in turn capable of collecting, concentrating and orchestrating the measurement and functioning of multiple dendrometers, consisting of:
An upper part of the casing (20);
A lower part of the casing (21);
A perforated cap(22);
A printed circuit board (23):
Sensors (24);
An antenna for long-distance wireless communication (25);
An external antennae connector (26);
A long-distance wireless communication module (27);
A switch (28) to switch the equipment on and off;
An USB communication interface (29);
A light sensor (30);
A sound sensor (31);
An air Temperature and humidity sensor (32); and
A smoke sensor (33).

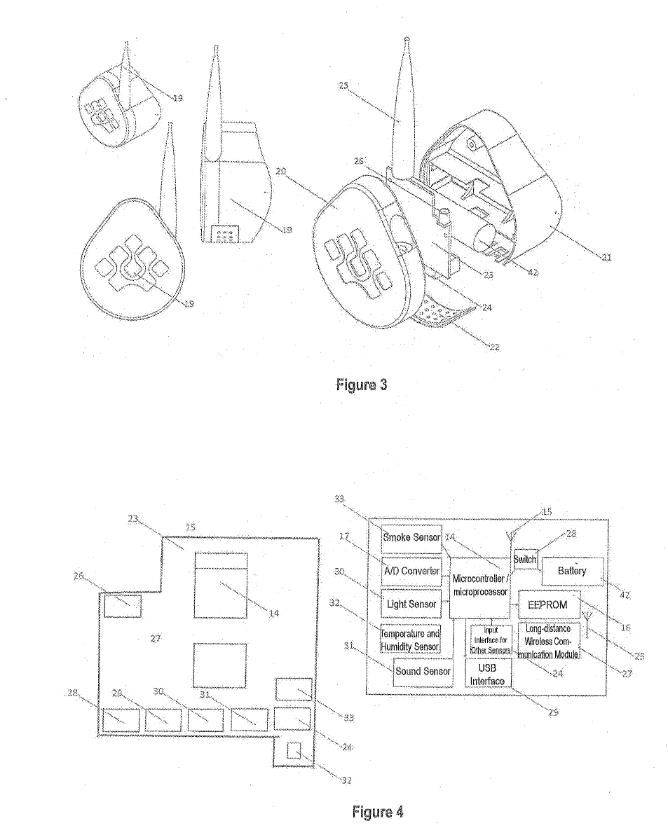

As can be seen in FIGS. 3 and 4, the concentrator (19) is responsible for receiving, storing and relaying the informations from the different dendrometers. This has a polymeric material casing that is divided into three parts—upper (20), lower (21) and perforated cap (22)—whose function is to protect the other components of the concentrator (19).

The printed circuit board (23) contains several sensors, such as a smoke sensor (33), a light sensor 30), an air temperature and humidity sensor (32) and a sound sensor (31), all connected to the microcontroller/microprocessor (4), which processes the signal received by the sensors, stores and relays via the long-distance wireless communication module (27).

The digital analog converter (17), when required, sends the digitized signal of the analog sensors to the microcontroller or microprocessor (14), which also receives the informations transmitted by the multiple dendrometers (1) through the antenna (15) and processes all the information obtained, storing them in the EEPROM memory (16).

The microcontroller or microprocessor (17) is also connected to a long-distance wireless communication module (27) that, via the long-range wireless communication antenna (25), relays the informations to the gateway (36) and to an USB communication interface (29), into which physical access the informations that are stored in the EEPROM memory (16) is allowed.

The inner circuit board (23) further contains an external antennae connector (26) that allows the use of antennas with different frequency ranges, or not, of the antennas already coupled in the present invention. There is also an input interface for other sensors (24), which allows various sensors to be coupled to the present invention.

The internal circuit (23) is powered by a high capacity battery (42) connected to a switch (28) to turn the equipment on and off.

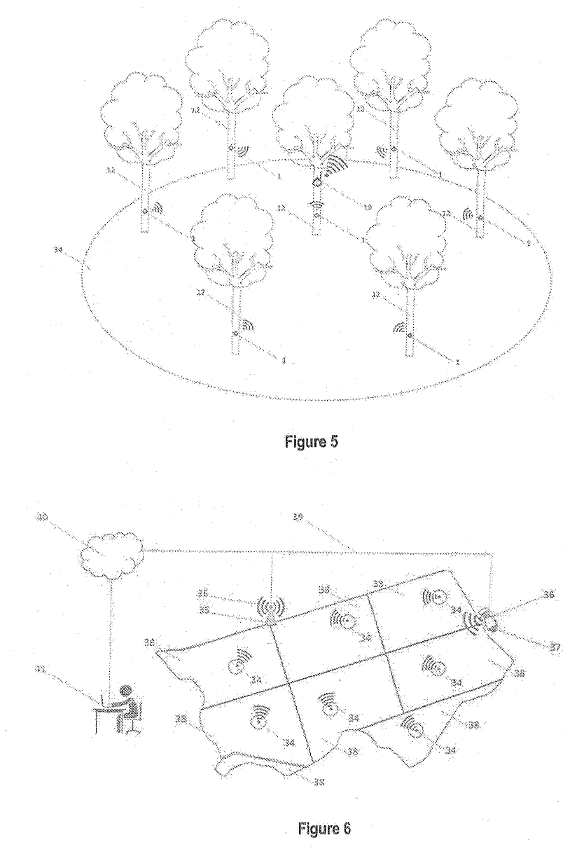

FIG. 5 demonstrates the arrangement of a system of the present invention in multiple trees (12), these being monitored by the dendrometers (1) connected to the concentrator (19) thereby forming a smart parcel (34).

FIG. 6, on the other hand, shows the population of a forest to be monitored and the multiple smart parcels (34) installed in the multiple plots of settlement (38), so that the systems installed in these parcels transfer the growth informations of environmental variables for a gateway (36) using radio frequency methodology, Long Range Radio (LoRa) or other Low-Power Area Network (LPWAN) methodology.

The gateway may be fixed and installed at the top of a tower (35) or mobile and installed in a round vehicle (37) to collect wireless data around the settlement whose interest is to monitor. In both cases, i.e., with the gateway installed in a tower (35) or in a vehicle (37), the informations are sent to the internet (39), wherein they will be stored in a cloud database (40) for further processing and access through a web platform (41).

Additionally, the present invention relates to a method for forest monitoring, comprising the steps of:

Step (a)—Installation of dendrometers (1) wherein dendrometers (1) are installed in the trees;
Step (b)—installation of concentrators (19) wherein the installation of the concentrators (19) in the trees is made;
Step (c)—Installation of multiple smart parcels (34) in the forest of interest;
Step (d)—Continuous monitoring of dendrometer variables
wherein dendrometer and other variables such as trunk diameter, morphological, biological status and health of the trees, air humidity and temperature, fire risk, level of weed competition, etc. are monitored;
Step (e)—Transfer of informations from the dendrometers (1) to the concentrators (19);
Step (f)—Transfer of informations from the concentrators to the gateway (36) and the Internet wherein it occurs at predefined intervals or on demand;
Step (g)—Storing the information in a cloud database (40);
Step (h)—Access to the informations remotely through a web platform (41).

The technicians skilled on the subject will appreciate the knowledge presented herein and can reproduce the invention in the shown embodiments and other variants encompassed in the scope of the attached claims.

1: A forest monitoring system comprising one or more dendrometers and a concentrator, connected to each other, which form a smart parcel, wherein the dendrometers each comprise a cable; a stressing band; a smaller gear; a gear; a rotary sensor; upper and lower parts of a casing; a cable fixing pin; a printed circuit board; a battery; a central pin; a microcontroller or microprocessor; an antenna; an EEPROM memory; a digital analog converter and a temperature sensor, wherein the concentrator comprises upper and lower parts of a casing; a perforated cap; a printed circuit board; sensors; an antenna for long-distance wireless communication; external antenna connectors; a long-distance wireless communication module; a switch to turn the equipment on and off; an USB communication interface; and light, sound temperature and air humidity and smoke sensors.

2: The system according to claim 1, wherein the cable is metallic or polymeric, and is adapted to surround a trunk of a tree to secure the device thereto, wherein the cable is adapted to unwind as the cable diameter is changed.

3: The system according to claim 1, wherein the cable is wrapped in a spool in the gear and is adapted to move the gear when the cable is unwound, which in turn moves the smaller gear to transfer movement to the rotary sensor, which can be resistive, capacitive, inductive or by pulse counting.

4: The system according to claim 1, wherein the stress band is a band spring that is adapted to exert sufficient stress on the cable producing stress for each dendrometer to remain attached to the trunk by static friction.

5: The system according to claim 1, wherein the printed circuit board is powered by the battery containing the rotary sensor and the temperature sensor responsible for correcting and compensating for the expansion of the system in relation to the temperature, where these are connected to the digital analog converter.

6: A system according to claim 1, wherein the microcontroller or microprocessor is adapted to process the digitized signals to store information in the EEPROM and to transmit the information using the antenna using radio frequency methodology, preferably Bluetooth Low Energy (BLE), Long Range Radio (LoRa) or other Low-Power Area Network (LPWAN) methodology, for the concentrator.

7: The system according to claim 1, wherein the upper part of the casing and the lower part of the casing are formed of polymer material with protection from ultraviolet rays and for preventing entry of insects and moisture.

8: A system according to claim 1, wherein the concentrator is adapted to be attached to the tree, using glue, nails, a spring or a brace, and comprises the printed circuit board containing the sensors connected to the microcontroller or microprocessor for sending the processed information via the long-distance wireless communication module to the gateway.

9: A system according to claim 1, wherein the microcontroller or microprocessor is adapted to receive information transmitted by the plurality of dendrometers via the antenna, processes them and stores them in the EEPROM memory.

10: A system according to claim 1, wherein the microcontroller or microprocessor is connected to the long-distance wireless communication module using of the long-range wireless communication antenna, adapted to relay the information to the gateway and a USB communication interface.

11: A system according to claim 1, wherein the internal circuit board comprises an external antenna connector and an input interface to other sensors, the latter being powered by a high capacity battery connected to a switch to turn the equipment on and off.

12: A system according to claim 1, wherein the gateway uses radio frequency methodology, Long Range Radio (LoRa) or other Low-Power Wide Area Network (LPWAN) methodology and is fixed and installed at the top of a tower or mobile and installed within a round vehicle.

13: A method for forest improvement comprising the steps of:
Step (a)—Installation of dendrometers in the trees;
Step (b)—Installation of concentrators in the trees;
Step (c)—Installation of multiple smart parcels in the forest of interest;
Step (d)—Continuous monitoring of dendrometer variables; wherein dendrometer and other variables selected from trunk diameter, morphological, biological status and health of the trees, air humidity and temperature, fire risk, level of weed competition, are monitored;
Step (e)—Transfer of information from the dendrometers to the concentrators;
Step (f)—Transfer of information from the concentrators to the gateway and the internet, wherein it occurs at predefined intervals or on demand;
Step (g)—Storing the information in a cloud database; and
Step (h)—Access to the informations remotely through a web platform.

14: The method according to claim 13, wherein the method is practiced using a forest monitoring system comprising one or more dendrometers and a concentrator, connected to each other, which form a smart parcel, wherein the dendrometers each comprise a cable; a stressing band; a smaller gear; a gear; a rotary sensor; upper and lower parts of a casing; a cable fixing pin; a printed circuit board; a battery; a central pin; a microcontroller or microprocessor; an antenna; an EEPROM memory; a digital analog converter and a temperature sensor, wherein the concentrator comprises upper and lower parts of a casing; a perforated cap; a printed circuit board; sensors; an antenna for long-distance wireless communication; external antenna connectors; a long-distance wireless communication module; a switch to turn the equipment on and off; an USB communication interface; and light, sound, temperature and air humidity and smoke sensors.

15: The method according to claim 13, wherein the cable is metallic or polymeric, and is adapted to surround a trunk of a tree to secure the device thereto, wherein the cable is adapted to unwind as the cable diameter is changed.

16: The method according to claim 13, wherein the cable is wrapped in a spool in the gear and is adapted to move the gear when the cable is unwound, which in turn moves the smaller gear to transfer movement to the rotary sensor, which can be resistive, capacitive, inductive or by pulse counting.

17: The method according to claim 13, wherein the stress band is a band spring that is adapted to exert sufficient stress on the cable producing stress for each dendrometer to remain attached to the trunk by static friction.

18: The method according to claim 13, wherein the printed circuit board is powered by the battery containing the rotary sensor and the temperature sensor responsible for correcting and compensating for the expansion of the system in relation to the temperature, where these are connected to the digital analog converter.

19: The method according to claim 13, wherein the microcontroller or microprocessor is adapted to process the digitized signals to store information in the EEPROM and to transmit the information using the antenna using radio frequency methodology, preferably Bluetooth Low Energy (BLE), Long Range Radio (LoRa) or other Low-Power Area Network (LPWAN) methodology, for the concentrator.

20: The method according to claim 13, wherein the upper part of the casing and the lower part of the casing are formed of polymer material with protection from ultraviolet rays and for preventing entry of insects and moisture.
